(19) United States
(12) Patent Application Publication
(10) Pub. No.: US 2020/0016345 A1
(43) Pub. Date: Jan. 16, 2020

(54) TIDAL INHALER ADAPTIVE DOSING

(71) Applicant: MICRODOSE THERAPEUTX, INC., Ewing, NJ (US)

(72) Inventors: Douglas WEITZEL, Hamilton, NJ (US); Henri AKOUKA, Mt. Laurel, NJ (US); Mark MORRISON, Basking Ridge, NJ (US)

(73) Assignee: MICRODOSE THERAPEUTX, INC., Ewing, NJ (US)

(21) Appl. No.: 16/496,021

(22) PCT Filed: Mar. 21, 2018
(86) PCT No.: PCT/US2018/023506

§ 371 Date: Sep. 20, 2019

Related U.S. Application Data
(60) Provisional application No. 62/475,079, filed on Mar. 22, 2017.

(51) Int. Cl. A61M 15/00

(52) U.S. Cl. CPC A61M 15/0085; A61M 2016/0018; A61M 15/0065

(57) ABSTRACT

A dry powder inhaler consisting of a first chamber having an orifice for holding a dry powder and a gas, and a second chamber directly connected to the first chamber by at least one passageway for receiving an aerosolized form of the dry powder from in the first chamber and delivering the aerosolized dry powder to a user. A pressure sensor monitors the pressure in the second chamber. A vibrator coupled to the first chamber aerosolizes the dry powder and cause the aerosolized powder to move through the passageway whereby to deliver the dry powder from the first chamber to the second chamber as an aerosolized dry powder. A vibrator control unit controls operation of the vibrator based on the monitored pressure in the second chamber and a dosing scheme in which the dosing time is determined by the volume of each inhalation.

### CROSS-REFERENCE TO PRIOR APPLICATIONS

This application claims priority to U.S. Provisional Patent Application No. 62/475,079, filed Mar. 22, 2017, which is hereby expressly incorporated by reference in its entirety.

### FIELD

The embodiments relate generally to the field of delivery of pharmaceuticals and drugs. Particular utility may be found in monitoring and regulating the delivery of a pharmaceutical or drug to a patient and will be described in connection with such utility, although other utilities are contemplated.

### BACKGROUND

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

Existing dry powder inhalers (DPIs) usually have a means for introducing the drug (active drug plus carrier) into a high velocity air stream. The high velocity air-stream is used as the primary mechanism for breaking up the cluster of micronized particles or separating the drug particles from the carrier. These devices present several problems and possess several disadvantages. First, conventional DPIs, generally being passive devices, contain no sensor or mechanism to regulate delivery of a dose of the dry powder formulation. Many conventional DPIs are designed to deliver a complete dose in one forced inhalation. Such disadvantages impact more severely affected patients by requiring them to sustain difficult breathing patterns through an inhaler with a moderate amount of flow resistance.

### SUMMARY

Embodiments described herein relate to methods, apparatuses, and/or systems for regulating the dosage of a pharmaceutical or drug delivered through an inhaler. In certain embodiments, the inhaler is capable of monitoring the patient's breathing so that it can release small amounts of drug formulation into the patient's inspiratory flow with each inhalation. In one embodiment, a dosing scheme utilizes a series of short bursts of drug delivery, or “shots,” delivered with the same number of successive inhalations to deliver a complete dose. It is desirable to reduce the amount of time as well as the number of successive inhalations required to deliver a complete dose. The reason for this is to reduce the amount of effort required by more severely affected patients who may have difficulty sustaining controlled breathing through an inhaler that has some amount of flow resistance.

In another embodiment, the inhaler is capable of utilizing an adaptive process, preferably an adaptive technique that minimizes the number of breaths, and therefore the time, required for the inhaler to deliver a full dose of dry powder drug formulation. Along with minimizing the number of breaths, the process is designed to ensure that a sufficient amount of chase air volume follows each inhalation of drug powder so that the drug can be effectively carried into the deeper regions of the lungs. In another embodiment, the inhaler utilizes an adaptive approach that minimizes drug delivery time and effort, and works effectively with different styles of breathing, such as tidal breathing or repeated forced inspiratory maneuvers (“pipe smoking”), or a combination of both. This multi-mode breathing capability is especially important as some patients are accustomed to forced inspiratory maneuvers from their use of metered dose or passive dry powder inhalers, while others are accustomed to tidal breathing from their use of nebulizers.

These methods, apparatuses, and/or systems provide significant advantages. First, monitoring the volume of the patient's breathing cycle to determine the piezoelectric activation time assists in ensuring that a certain amount of chase volume is available, while minimizing the number of inhalations needed. This is particularly advantageous especially when combined with utilizing a post-peak drop-off in flow rate as a safety mechanism for preventing exhalation of drug powder. Second, monitoring flow rate of the patient's breathing cycle serves as a safety mechanism to end the shot in the event that the breath is smaller than the assumed volume. In these embodiments, the piezoelectric activation time, and thus the dose delivery time associated with a single shot, may be increased if the current breath is larger than the previous breath to compensate for breath-to-breath differences, thereby minimizing the total dosing session time. In addition, the total dosing time under most adult breathing situations is significantly reduced, especially when stronger inhalation is present. This encourages more effective inspiratory effort by rewarding the patient with a shorter treatment time, while at the same time accommodating weaker and/or more variable breathing patterns for more severe cases.

Various other aspects, features, and advantages will be apparent through the detailed description and the drawings attached hereto. It is also to be understood that both the foregoing general description and the following detailed description are exemplary and not restrictive of the scope of the embodiments. As used in the specification and in the claims, the singular forms of “a”, “an”, and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

FIGS. 1A-C show a perspective view of an inhaler, in accordance with one or more embodiments.

FIG. 2 shows a functional block diagram of an inhaler control unit, in accordance with one or more embodiments.

FIGS. 3 and 4 show flowcharts of methods of delivering a dose of a drug with an inhaler, in accordance with one or more embodiments.

FIGS. 5-8 show graphs depicting breath patterns of patients utilizing the dosing techniques, in accordance with one or more embodiments.

### DETAILED DESCRIPTION

In the following description, for the purposes of explanation, numerous specific details are set forth in order to provide a thorough understanding of the embodiments. It will be appreciated, however, by those having skill in the art that the embodiments may be practiced without these specific details or with an equivalent arrangement. In other instances, well-known structures and devices are shown in block diagram form in order to avoid unnecessarily obscuring the embodiments of the invention.

The present embodiments relate to a device for administering medicament as a dry powder for inhalation by a subject. Some embodiments of the device may be classified as a dry powder inhaler (DPI). Some embodiments of the device may also be classified as a dry powder nebulizer (as opposed to a liquid nebulizer), particularly when tidal breathing is used to deliver dry powder medicament over multiple inhalations. The device may be referred to herein interchangeably as a “device” or an “inhaler,” both of which refer to a device for administering medicament as a dry powder for inhalation by a subject, preferably over multiple inhalations, and most preferably when tidal breathing is used. “Tidal breathing” preferably refers to inhalation and exhalation during normal breathing at rest, as opposed to forceful breathing.

Structure and Operation of an Inhalation Device

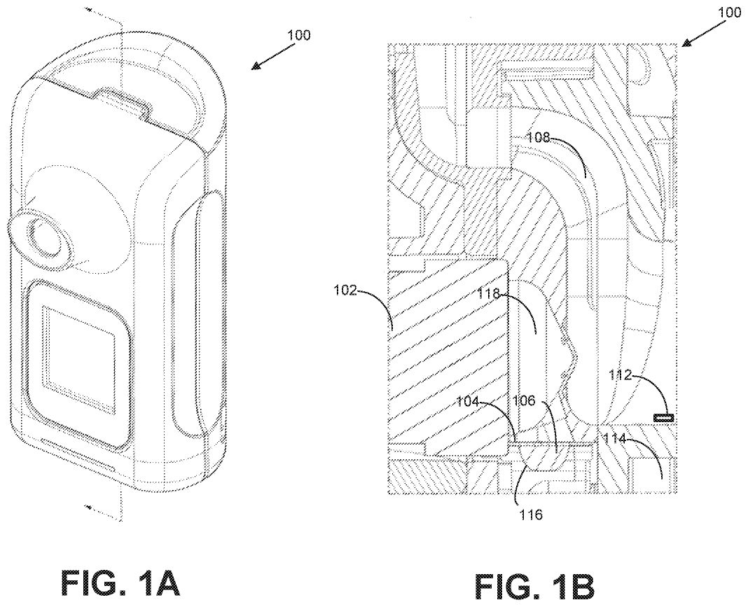

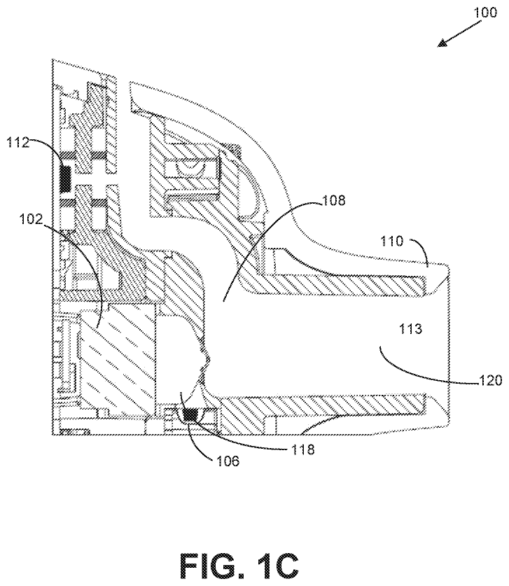

FIGS. 1A-C show an inhaler 100 configured to receive a user's inhale through the mouthpiece of the device, preferably via tidal breathing, and deliver a dose of medicament over a plurality of consecutive inhalations. In one embodiment illustrated in FIGS. 1A-C, the inhaler 100 may be configured to activate transducer 102 more than once to deliver a complete pharmaceutical dose from a drug cartridge 104 to a user. During operation, when the user inhales through the mouthpiece, air is drawn into the inhaler's air inlet, through an air flow conduit in the device, and out of the mouthpiece into the user's lungs; as air is being inhaled through the air flow conduit, dry powder medicament is expelled into the airflow pathway and becomes entrained in the user's inhaled air. Thus, the air flow conduit preferably defines an air path from the air inlet to the outlet (i.e., the opening that is formed by the mouthpiece). Each breath cycle includes an inhalation and an exhalation, i.e., each inhalation is followed by an exhalation, so consecutive inhalations preferably refer to the inhalations in consecutive breath cycles. After each inhalation, the user may either exhale back into the mouthpiece of the inhaler, or exhale outside of the inhaler (e.g., by removing his or her mouth from the mouthpiece and expelling the inhaled air off to the side). In one embodiment, consecutive inhalations refer to each time a user inhales through the inhaler which may or may not be each time a patient inhales their breath.

In one embodiment, the inhaler 100 may contain a plurality of pre-metered doses of a dry powder drug composition comprising at least one medicament, wherein each individual dose of the plurality of pre-metered doses is inside a drug cartridge 104, such as a blister 106. As used herein, a blister 106 may include a container that is suitable for containing a dose of dry powder medicament. Preferably, a plurality of blisters may be arranged as pockets on a strip, i.e., a drug cartridge. According to a preferred embodiment, the individual blisters may be arranged on a peelable drug strip or package, which comprises a base sheet in which blisters are formed to define pockets therein for containing distinct medicament doses and a lid sheet which is sealed to the base sheet in such a manner that the lid sheet and the base sheet can be peeled apart; thus, the respective base and lid sheets are peelably separable from each other to release the dose contained inside each blister. The blisters may also be preferably arranged in a spaced fashion, more preferably in progressive arrangement (e.g. series progression) on the strip such that each dose is separately accessible.

FIGS. 1A-C shows an inhaler 100 configured to activate the transducer 102 more than once to deliver a complete pharmaceutical dose from a single blister 106 to a user. In one embodiment, the inhaler 100 may include an air flow conduit 108 configured to allow air to travel through the inhaler 100 when a user inhales through a mouthpiece 110. In one embodiment, the inhaler 100 may include an inhalation sensor 112 configured to detect airflow through the air flow conduit 108 and send a signal to a controller 114 when airflow is detected. In one embodiment, the controller 114 may be configured to activate a drug strip advance mechanism 116, when a flow of air is detected by the sensor 112 (in some cases, when a first flow of air is detected). The drug strip advance mechanism 116 may be configured to advance a drug strip 104 a fixed distance (e.g., the length of one blister) such that the blister 106 is in close proximity to (or in one embodiment adjacent to or substantially adjacent to) a dosing chamber 118, for example. A membrane (not shown) may be configured to cover an open end of the dosing chamber 118 in one embodiment. In one embodiment, transducer 102 may confront the membrane of the dosing chamber 118. In one embodiment, the controller 114 may be configured to activate a transducer 102 when an activation event is detected. In one embodiment, detection of multiple inhalations may be required to trigger activation of transducer 102. For example, controller 114 may be configured to activate a transducer 102 when a flow of air is detected by the sensor 112 (in some cases, when a subsequent flow of air is detected, e.g., second, third, or later). The transducer 102 may be configured to vibrate, thereby vibrating the membrane, to aerosolize and transfer pharmaceutical from the blister 106 into the dosing chamber 118. In one embodiment, the vibration of the transducer 102 also delivers the aerosolized pharmaceutical into the dosing chamber 118, through the exit channel 120, and to a user through mouthpiece 110.

The transducer 102 may be a piezoelectric element made of a material that has a high-frequency, and preferably, ultrasonic resonant vibratory frequency (e.g., about 15 to 50 kHz), and is caused to vibrate with a particular frequency and amplitude depending upon the frequency and/or amplitude of excitation electricity applied to the piezoelectric element. Examples of materials that can be used to comprise the piezoelectric element may include quartz and polycrystalline ceramic materials (e.g., barium titanate and lead zirconate titanate). Advantageously, by vibrating the piezoelectric element at ultrasonic frequencies, the noise associated with vibrating the piezoelectric element at lower (i.e., sonic) frequencies can be avoided.

In some embodiments, the inhaler 100 may comprise an inhalation sensor 112 (also referred to herein as a flow sensor or breath sensor) that senses when a patient inhales through the device; for example, the sensor 112 may be in the form of a pressure sensor, air stream velocity sensor or temperature sensor. According to one embodiment, an electronic signal may be transmitting to controller 114 contained in inhaler 100 each time the sensor 112 detects an inhalation by a user such that the dose is delivered over several inhalations by the user. For example, sensor 112 may comprise a conventional flow sensor which generates electronic signals indicative of the flow and/or pressure of the air stream in the air flow conduit 108, and transmits those signals via electrical connection to controller 114 contained in inhaler 100 for controlling actuation of the transducer 102 based upon those signals and a dosing scheme stored in memory (not shown). Preferably, sensor 112 may be a pressure sensor. Non-limiting examples of pressure sensors that may be used in accordance with embodiments may include a microelectromechanical system (MEMS) pressure sensor or a nanoelectromechanical system (NEMS) pressure sensor herein. The inhalation sensor may be located in or near an air flow conduit 108 to detect when a user is inhaling through the mouthpiece 110.

Preferably, the controller 114 may be embodied as an application specific integrated circuit chip and/or some other type of very highly integrated circuit chip. Alternatively, controller 114 may take the form of a microprocessor, or discrete electrical and electronic components. As will be described more fully below, the controller 114 may control the power supplied from conventional power source 154 (e.g., one or more D.C. batteries) to the transducer 102 according to the signals received from sensor 112 and a dosing scheme stored in memory (not shown). The power may be supplied to the transducer 102 via electrical connection between the vibrator and the controller 114. In one embodiment, an electrical excitation may be applied to the transducer 102 generated by the controller 114 and an electrical power conversion sub-circuit (not shown) converts the DC power supply to high-voltage pulses (typically 220 Vpk-pk) at the excitation frequency.

Memory may include non-transitory storage media that electronically stores information. The memory may include one or more of optically readable storage media, electrical charge-based storage media (e.g., EEPROM, RAM, etc.), solid-state storage media (e.g., flash drive, etc.), and/or other electronically readable storage media. The electronic storage may store dosing algorithms, information determined by the processors, information received from sensors, or other information that enables the functionality as described herein.

In operation, blister 106 may be peeled open and placed adjacent to an opening in the dose chamber 118 in the manner described previously. The user inhales air through the air flow conduit 108 and air stream is generated through air flow conduit 108. The flow and/or pressure of inhalation of the air stream may be sensed by a sensor 112 and transmitted to controller 114, which supplies power to transducer 102 based according to the signals and a stored dosing scheme. Controller 114 may adjust the amplitude and frequency of power supplied to the transducer 102 until they are optimized for the best possible deaggregation and suspension of the powder from the capsule into the air stream.

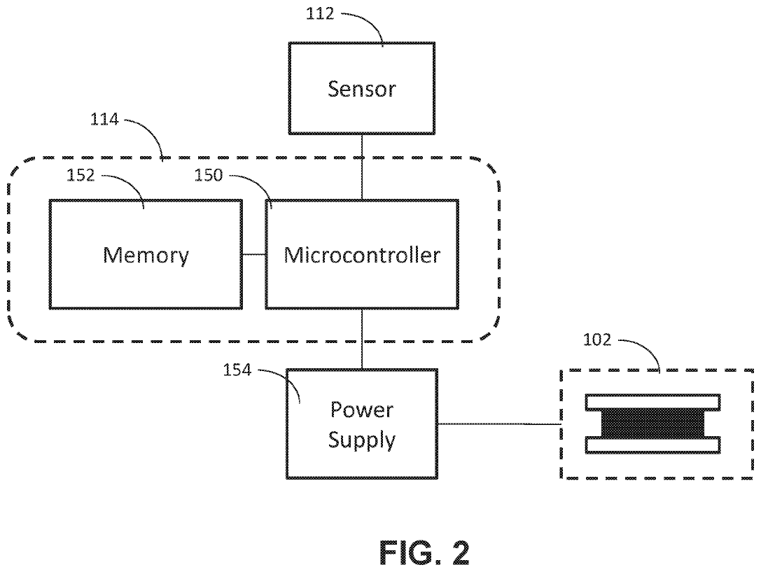

Turning to FIG. 2, the various functional components and operation of the controller 114 will now be described. As will be understood by those skilled in the art, although the functional components shown in FIG. 2 are directed to a digital embodiment, it should be appreciated that the components of FIG. 2 may be realized in an analog embodiment.

Inhalation Detection

In one embodiment, controller 114 may include a microcontroller 150 for controlling the power 152 supplied to transducer 102 based on the signals received from sensor 112 and a dosing scheme stored in memory 152.

In one embodiment, sensor 112 may be configured to transmit a signal of the detection of an inhalation after a detection event has occurred. The detection event may include a select number of dosing breaths (e.g., 1, 2, 3, 4 or five preliminary dosing breaths), a fixed quantity of dosing breaths (e.g., a total volume or mass of air is breathed) or a selected threshold is met. In another embodiment, after the inhaler 100 is turned on, the pressure in air flow conduit 108 may be monitored by sensor 112 to determine when the user starts breathing. For example, microcontroller 150 may determine whether the user is breathing by calculating the rate of change of pressure within air flow conduit 108. The rate of change of pressure is then compared to predetermined upper and lower limits to ensure an appropriate rate of change has occurred. These upper and lower limits are utilized to reject ambient pressure disturbances in the environment, such as sudden changes in altitude, use of the tidal inhaler in a moving vehicle, opening or closing of doors, fast-moving weather systems, etc. that could results in false triggers due to the high sensitivity of the pressure sensor. When the rate of change is between the predetermined upper and lower limit, for the first time, microcontroller 150 may average a predetermined number of pressure samples prior to that point to calculate a baseline pressure.

In some embodiments, once the start of inhalation has been detected, microcontroller 150 may accumulate pressure values scaled to volumetric flow rate units to calculate an inhalation volume. As breathing continues, the accumulation of scaled pressure values may be stopped in response to the pressure values crossing the zero point into a positive range where exhalation begins. In one embodiment, microcontroller 150 may compare the inhalation volume to a predetermined threshold to determine if the volumetric value is detected as an appropriate inhalation volume. If the inhalation volume exceeds the predetermined threshold, the microcontroller 150 may detect a start of inhalation for a next breath cycle of the user. If the inhalation volume does not exceed the predetermined threshold, the current breath is ignored and determination of the inhalation volume for the first breath cycle of a user is repeated.

In some embodiments, when the start of the next inhalation is detected as an appropriate rate of change of pressure, and the relative pressure exceeds a predetermined triggering threshold, microcontroller 150 may generate a dosing trigger. In response to the dosing trigger being generated in a second breath cycle, microcontroller 150 may advance the drug strip into position on transducer 102. In response to the dosing trigger being generated for any subsequent breath cycle, microcontroller 150 may activate transducer 102 according to a dosing scheme. For example, in some embodiments, the dosing scheme may activate the transducer 102 for a predetermined duration of time. In some embodiments, the entire dosing scheme may require ten valid subsequent breath cycles. For example, the dosing trigger may activate the transducer 102 for 100 milliseconds for the third through sixth breath cycles and may activate the transducer 102 for 300 milliseconds for the seventh through tenth breath cycles (a total activation time of 1.6 seconds). It should be appreciated that the number of breath cycles and the predetermined duration of time for the dosing scheme are not limiting and may vary based on the characteristics of the drug and/or user.

It should be appreciated that the dosing session may be repeated for one or more subsequent breath cycles to ensure that the relative pressure in the air flow conduit 108 is above the predetermined triggering threshold before the dosing trigger is generated for that particular breath cycle. In the event that the start of an inhalation of a breath cycle is not detected within a predetermined time interval following the generation of the dosing trigger, the dosing session may be reset. In one embodiment, if the dosing session is reset, the dosing scheme may resume on the breath cycle that was not detected. For example, if the start of inhalation of the sixth breath cycle was not detected within the predetermined time interval, the dosing session will reset and a new baseline pressure may be calculated. However, rather than repeat the triggering events already performed, the dosing scheme may continue on the sixth breath cycle.

Adaptive Triggering

In another embodiment, controller 114 may control the power 154 supplied to the transducer 102 based on the signals received from sensor 112 and an adaptive dosing scheme stored in memory 152. Similar to the previously described inhalation detection method, microcontroller 150 may determine the start of inhalation using the rate of change of pressure, and then calculate the inhalation volume for a first breath cycle. When the volume exceeds a predetermined threshold, microcontroller 150 may detect the start of inhalation and calculate the inhalation volume for a second breath cycle.

In some embodiments, microcontroller 150 may utilize the calculated volume of the first and second inhalation to determine the dosing shot volume for the next inhalation assuming that the volume will be similar for every breath. The dosing shot volume may, for example, be based on some fixed percentage, such as 40% of the total volume measured. It should be appreciated that the dosing shot value may be adjusted based on a number of factors including, but not limited to, the inhalation volume for each breath cycle, the dose amount, the minimum number of doses, etc.

In some embodiments, similar to the previously described inhalation detection technique, microcontroller 150 may activate transducer 102 based on having reached a minimum volume in the previous breath cycle combined with reaching the inhalation flow rate threshold provided that the rate of change of pressure was within the appropriate range. In some embodiments, transducer 102 may be activated in a single burst or rapidly repeating shorter bursts. The advantage of the shorter bursts is that the drug powder would be introduced into the patient's inspiratory flow at a slower rate to improve deposition in the lung, especially if the patient is inhaling with a relatively high flow rate. It should be appreciated that microcontroller 150 may determine which of the two activation methods is used based on the measured flow rate for each inhalation.

During the inhalation, controller 114 may deactivate transducer 102 in response to the calculated volume equaling the dosing shot volume determined from the previous inhalations. It should be appreciated that at this point, all remaining inhaled air serves as chase volume for the drug dispensed during that shot. Also during the inhalation, microcontroller 150 may monitor the flow rate to determine when the flow rate starts to decrease after reaching a peak (or sustained) value. If this occurs before the dosing shot volume is reached, microcontroller 150 may deactivate transducer 102 as a safety mechanism to ensure that some minimum chase volume can pass. Optionally, if the flow rate is still high after the shot volume has been reached, microcontroller 150 may continue operation of the transducer 102 until the flow rate starts to decrease. This latter option would help to shorten the dosing time, but could also result in much smaller chase volumes. In some embodiment, a method for determining when the peak inhalation rate has passed may include some hysteresis during the high flow portion of the inhalation to avoid ending the shot prematurely. For example, rate or magnitude of changes in flow rate and/or volume could be used as inputs to determine the peak inhalation rate.

It will be understood by persons having ordinary skill in the art that the dosing session may be repeated for one or more subsequent breath cycles to ensure that the dosing session is complete. In one embodiment, the dosing session may end when the accumulated total dosing shot duration (piezoelectric element activation time) equals a predetermined total time. As an example, the end of a dosing session may occur when the total dosing shot duration, in this case, 1.6 seconds [equivalent to the total shot duration used in the first embodiment described above of (4 shots×100 ms per shot)+(4 shots×300 ms per shot)], is equal to a predetermined total time.

Exemplary Flowcharts

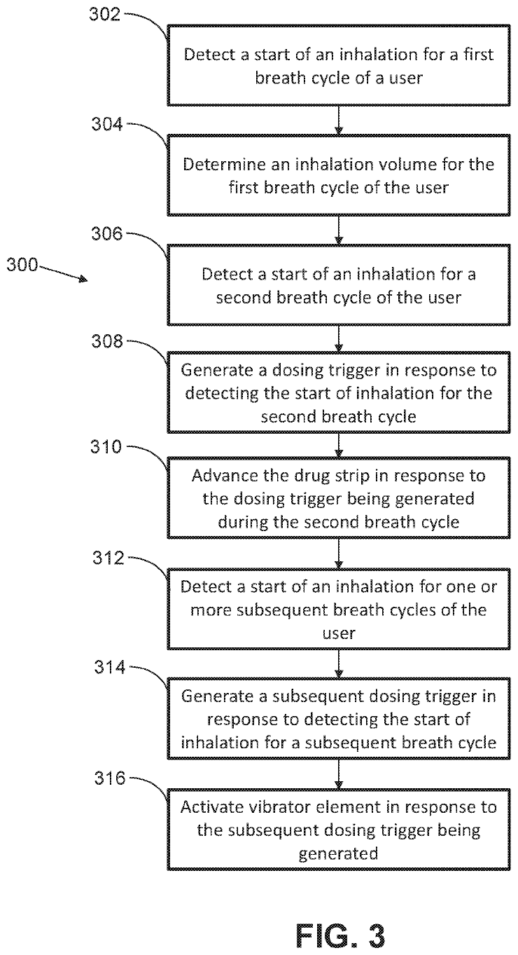

FIG. 3 illustrates a flowchart of an exemplary method 300 of delivering a dose of a drug with an inhaler, in accordance with one or more embodiments.

In an operation 302, a start of an inhalation of a first breath cycle of a user is detected. As an example, after the inhaler is turned on, the pressure in the flow channel is monitored to determine when the user starts an inhalation. This is determined by calculating the rate of change of pressure within the flow channel. The rate of change of pressure is then compared to predetermined upper and lower limits to ensure an appropriate rate of change has occurred. When the rate of change is between the predetermined upper and lower limit, for the first time, an average of a predetermined number of pressure samples prior to that point are utilized to calculate a baseline pressure. If the rate of change is not within the predetermined upper and lower limits, the current breath cycle is ignored and detection of the start of an inhalation for the first breath cycle of the user is repeated.

In an operation 304, an inhalation volume of the first breath cycle of the user is determined. As an example, after the start of an inhalation of the first breath cycle is detected, pressure values are collected until the pressure values crosses the zero point into a positive range where exhalation of the first breath cycle begins. The pressure values are converted to flow rate values knowing the flow resistance of the flow channel 108 according to the relationship Flow Rate=(Pressure Drop)^{1/2}/Flow Resistance. The flow rate values are numerically integrated with respect to time to calculate an inhalation volume. In one embodiment, the inhalation volume is compared to a predetermined threshold to determine if the volumetric value is detected as an appropriate amount of inhalation volume. If the inhalation volume exceeds the predetermined threshold, a start of inhalation of a second breath cycle of the user is determined. If the inhalation volume does not exceed the predetermined threshold, the current breath is ignored and operations 302 and 304 are repeated.

In an operation 306, a start of an inhalation of a second breath cycle of the user is detected. As an example, similar to detection of the start of an inhalation for the first breath cycle, the pressure in the flow channel is monitored to determine when the user starts an inhalation. The rate of change in pressure is compared to the predetermined upper and lower limit to determine if an appropriate change of pressure has occurred. If the rate of change is not within the upper and lower limits, the current breath cycle is ignored and detection of the start of an inhalation for the second breath cycle of the user is repeated.

In an operation 308, a dosing trigger is generated in response to the start of inhalation for a second breath cycle being detected. As an example, once the start of an inhalation for the second breath cycle of the user is detected, the relative pressure in the flow channel is compared to a predetermined triggering threshold. If the relative pressure in the flow channel is above the predetermined triggering threshold, a dosing trigger is generated. If the relative pressure in the flow channel does not exceed the predetermined triggering threshold, the breath cycle is ignored and detection of the start of an inhalation for the second breath cycle of the user in operation 306 is repeated.

In an operation 310, in response to the dosing trigger being generated during the second breath cycle, the drug strip is advanced. For example, in one embodiment, the generated dosing trigger advances the drug cartridge during the second breath cycle.

In an operation 312, a start of an inhalation of one or more subsequent breath cycles of the user is detected. Similar to operation 306, the pressure in the flow channel is monitored to determine when the user starts an inhalation. The rate of change is compared to the predetermined upper and lower limit to determine if an appropriate change of pressure has occurred. If the rate of change is not within the predetermined upper and lower limits, the current breath cycle is ignored and detection of the start of an inhalation for the subsequent breath cycle of the user is repeated.

In an operation 314, a subsequent dosing trigger is generated in response to the start of inhalation for a subsequent breath cycle being detected. As an example, similar to operation 308, once the start of an inhalation for the subsequent breath cycle of the user is detected, the relative pressure in the flow channel is compared to a predetermined triggering threshold. If the relative pressure in the flow channel is above the predetermined triggering threshold, a subsequent dosing trigger is generated. If the relative pressure in the flow channel does not exceed the predetermined triggering threshold, the current breath cycle is ignored and detection of the start of an inhalation for another subsequent breath cycle of the user in operation 312 is repeated.

In an operation 316, the piezoelectric element is activated according to a dosing scheme in response to the subsequent dosing trigger being generated during one or more subsequent breath cycle. For example, in one embodiment, the generated subsequent dosing trigger may activate the piezoelectric element for a predetermined duration of time according to the predetermined dosing scheme. In one embodiment, the entire dosing scheme may require ten valid subsequent breath cycles. For example, the dosing trigger may activate the piezoelectric element for 100 milliseconds for the third through sixth breath cycles and the dosing trigger may activate the piezoelectric element for 300 milliseconds for the seventh through tenth breath cycles (a total activation time of 1.6 seconds). It should be appreciated that the number of breath cycles and the predetermined duration of time for the dosing scheme are not limiting and may vary based on the characteristics of the drug and/or user. For example, the dosing trigger may activate the piezoelectric element for anywhere from about 25 to about 250, or from about 50 to about 200, or from about 65 to about 145, or from about 75 to about 125, or about 100 milliseconds for the third through sixth breath cycles, and the dosing trigger may activate the piezoelectric element for anywhere from about 125 to about 650, or from about 175 to about 500, or from about 225 to about 400, or from about 250 to about 350, or about 300 milliseconds for the seventh through tenth breath cycles, or any values therebetween.

It will be appreciated and understood by those having ordinary skill in the art that operations 312 and 314 may be repeated for one or more subsequent breath cycles to ensure that the relative pressure in the flow chamber is above the predetermined triggering threshold before the dosing trigger is generated for that particular breath cycle. In the event that the start of an inhalation of a breath cycle is not detected within a predetermined time interval following the generation of the dosing trigger, the dosing session will reset and return to operation 302. If the dosing session is reset, the dosing scheme may resume on the breath cycle which not detected.

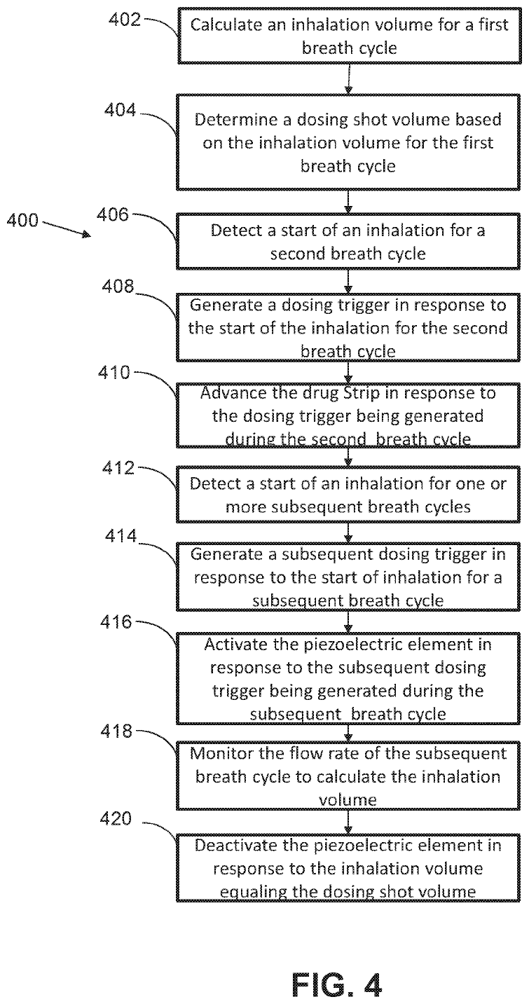

FIG. 4 illustrates a flowchart of a method 400 that is exemplary for delivering an adaptive dose of a drug with an inhaler, in accordance with one or more embodiments.

In an operation 402, an inhalation volume of a first breath cycle of a user is calculated. As an example, after the inhaler is turned on, the pressure in the flow channel is monitored to determine when the user starts an inhalation of the first breath cycle. This is determined by calculating the rate of change of pressure within the flow channel. When the rate of change is between a predetermined upper and lower limit, for the first time, an average of a predetermined number of pressure samples prior to that point are utilized to calculate a baseline pressure. If the rate of change of pressure is not within the predetermined upper and lower limits, the breath cycle is ignored and detection of the start of an inhalation for the first breath cycle of the user is repeated. After the start of an inhalation is detected, pressure values are collected until the pressure values crosses the zero point into a positive range where exhalation begins. The pressure values are converted to flow rate values knowing the flow resistance of the flow channel 108 according to the relationship Flow Rate=(Pressure Drop)^{1/2}/Flow Resistance. The flow rate values are numerically integrated with respect to time to calculate an inhalation volume for the first breath cycle. In one embodiment, the inhalation volume is compared to a predetermined threshold to determine if the volumetric value is detected as an appropriate amount of inhalation volume. If the inhalation volume exceeds the predetermined threshold, an inhalation volume for a second breath cycle of the user is calculated. If the inhalation volume does not exceed the predetermined threshold, the current breath cycle is ignored and determination of the inhalation for inhalation volume for the first breath cycle of a user in operation 402 is repeated.

In an operation 404, a dosing shot volume is determined based on the inhalation volume for the first breath. For example, the calculated inhalation volume for the first breath cycle is utilized to determine the dosing shot volume for each subsequent breath cycle. In one embodiment, the dosing shot volume may be based on a fixed percentage of the total inhalation volumes for the first and second breath cycles, such as from about 25 to about 75%, or from about 35 to about 65%, or from about 40 to about 50% of the total inhalation volumes calculated. It should be appreciated that the dosing shot volume for the dosing scheme is not limiting, and may vary based on the characteristics of the drug and/or user. Using the guidelines provided herein, those skilled in the art will be capable of developing operations that effectively determine the dosing shot volume for the dosing scheme using various characteristics of the drug and/or user.

In an operation 406, a start of an inhalation of a second breath cycle of the user is detected. For example, the pressure in the flow channel is monitored to determine when the user starts an inhalation of the third breath cycle. The rate of change is compared to the predetermined upper and lower limit to determine if an appropriate change of pressure has occurred. If the rate of change is not within the upper and lower limits, the third breath cycle is ignored and detection of the start of an inhalation for the breath cycle of the user in operation 408 is repeated.

In an operation 408, a dosing trigger is generated in response to the start of inhalation for a third breath cycle being detected. As an example, once the start of an inhalation of the third breath cycle of the user is detected, the relative pressure in the flow channel is compared to a predetermined triggering threshold. If the relative pressure in the flow channel is above the predetermined triggering threshold, a dosing trigger is generated. If the relative pressure in the flow channel does not exceed the predetermined triggering threshold, the breath cycle is ignored and detection of the start of an inhalation for the third breath cycle of the user in operation 408 is repeated.

In an operation 410, a drug strip is advanced in response to the dosing trigger being generated during the second breath cycle. For example, in one embodiment, the generated dosing trigger advances the drug strip during the second breath cycle.

In an operation 412, a start of an inhalation of one or more subsequent breath cycles of the user is detected. Similar to operation 408, the pressure in the flow channel is monitored to determine when the user starts an inhalation. The rate of change is compared to the predetermined upper and lower limit to determine if an appropriate change of pressure has occurred. If the rate of change of pressure is not within the predetermined upper and lower limits, the subsequent breath cycle is ignored and detection of the start of an inhalation for the subsequent breath cycle of the user is repeated.

In an operation 414, a subsequent dosing trigger is generated in response to the start of inhalation for a subsequent breath cycle being detected. As an example, similar to operation 410, once the start of an inhalation for the subsequent breath cycle of the user is detected, the relative pressure in the flow channel is compared to a predetermined triggering threshold. If the relative pressure in the flow channel is above the predetermined triggering threshold, a subsequent dosing trigger is generated. If the relative pressure in the flow channel does not exceed the predetermined triggering threshold, the subsequent breath cycle is ignored and detection of the start of an inhalation for another subsequent breath cycle of the user in operation 414 is repeated.

In an operation 416, the piezoelectric element is activated in response to the dosing trigger being generated during one or more subsequent breath cycles. For example, the piezoelectric element may be activated in a single burst or rapidly repeating bursts for each subsequent breath cycle. In one embodiment, the piezoelectric element activation may be determined based on the measured flow rate for each breath cycle. For example, the dosing scheme for breath cycles with a lower flow rate may utilize a single burst while breath cycles with a higher flow rate may utilize rapid bursts.

In an operation 418, the flow rate of the subsequent breath cycle is monitored to calculate the inhalation volume during the subsequent breath cycle. For example, once the start of the of the subsequent inhalation is detected, pressure values are collected until the pressure values crosses the zero point into a positive range where exhalation has begun. The pressure values are converted to flow rate values knowing the flow resistance of the flow channel 108 according to the relationship Flow Rate=(Pressure Drop)^{1/2}/Flow Resistance. The flow rate values are numerically integrated with respect to time to calculate a subsequent inhalation volume.

In an operation 420, in response to the subsequent inhalation volume is equal to the dosing shot volume, the piezoelectric element is deactivated. As an example, in response to the calculated subsequent inhalation volume equaling the dosing shot volume, the piezoelectric element is de-activated. It should be appreciated at this point all remaining inhaled air serves as chase volume for the drug dispensed during that shot. In one embodiment, the dosing session may be optimized based on monitored flow rate during inhalation of the subsequent breath cycle. For example, if the monitored flow rate begins to decrease after reaching a peak or sustained value before the dosing shot volume is reached, the piezoelectric element may be de-activated as a safety mechanism to ensure that some minimum chase volume can pass. In another embodiment, if the monitored flow rate is high after the shot volume has been reached, activation of the piezoelectric element may continue until the monitored flow rate starts to decrease. It should be appreciated that this may shorten the dosing time, but may result in much smaller chase volumes.

It will be appreciated that operations 414 through 420 may be repeated for one or more subsequent breath cycles to ensure that the accumulated total actual shot duration (piezo activation time) completes a predetermined dosing scheme. In one embodiment, the entire dosing scheme may be based on a total dosing time such as from about 0.5 to about 5 seconds, or from about 0.75 to about 4 seconds, or from about 1 to about 2.5 seconds, or about 1.6 seconds, or any value therebetween. In this case, the number of subsequent breath cycles would be based on the duration of activation time of the piezoelectric element during each of those subsequent breath cycles. Once the activation time of the piezoelectric element equals the totaled actual shot duration, the dosing session is complete.

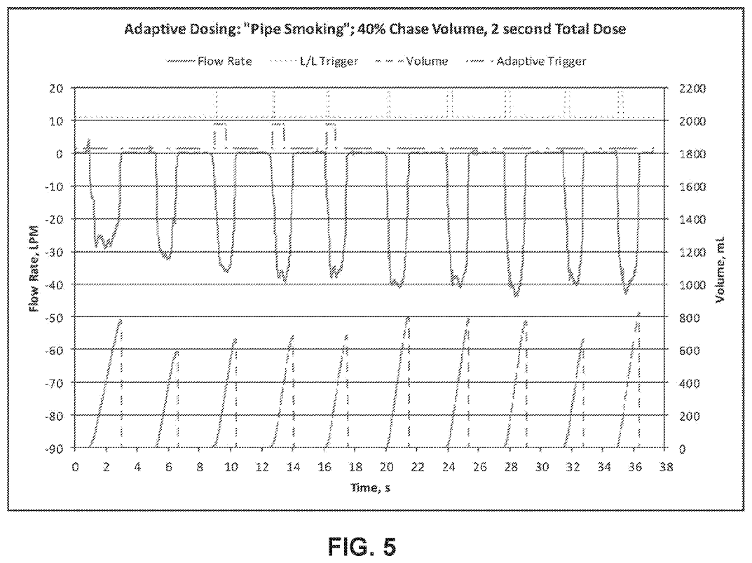

According to an exemplary embodiment, FIG. 5 illustrates a breathing pattern collected from a COPD patient utilizing an inhaler using the adaptive triggering technique described herein. The breathing style for the patient involved forced inspiratory maneuvers, indicative of strong, steady pipe smoking, where exhalations were not passed through the inhaler. As shown in FIG. 5, the patient's breathing cycles comprise a strong, steady flow rate and volume as depicted by the bottom two lines in the graph. Due to the adaptive triggering technique, a significant reduction in the number inhalations required to complete delivery of the dose is reduced from eight, as is the case when using a non-adaptive (fixed) trigger technique, to three when using the inventive adaptive triggering technique, as depicted in the line second from the top.

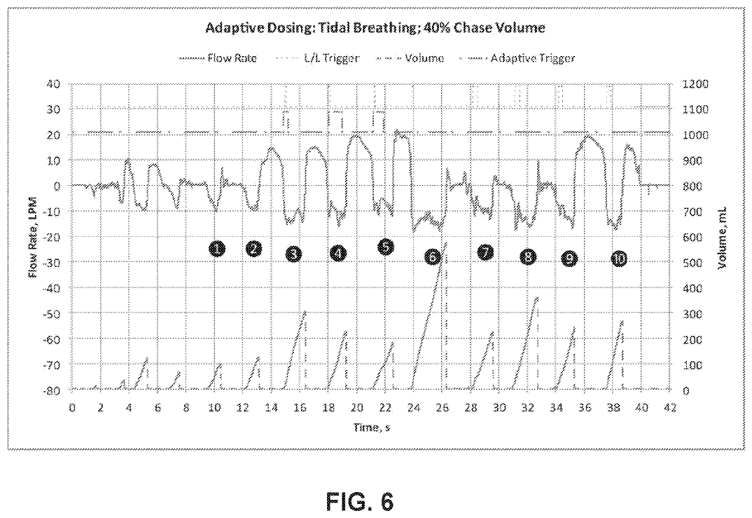

According to an exemplary embodiment, FIG. 6 illustrates a breathing pattern collected from another COPD patient utilizing an inhaler using the adaptive triggering technique. The breathing style of this patient included weak, irregular tidal breathing, with 40% chase volume. As shown in FIG. 6, the patient's breathing cycles are weak in flow rate and irregular in volume as depicted by the bottom two lines in the graph. However, due to the adaptive triggering technique, a significant reduction in the number of inhalations required to complete delivery of the dose is reduced from eight, as is the case when using the non-adaptive (fixed) trigger technique, to three when using the inventive adaptive triggering technique, as shown in the line second from the top. In this example, the first shot in the third breath cycle is shorter than necessary because the previous inhalation was small. In addition, the chase volume required in the second shot in the fourth breath cycle was not met because the previous breath was larger.

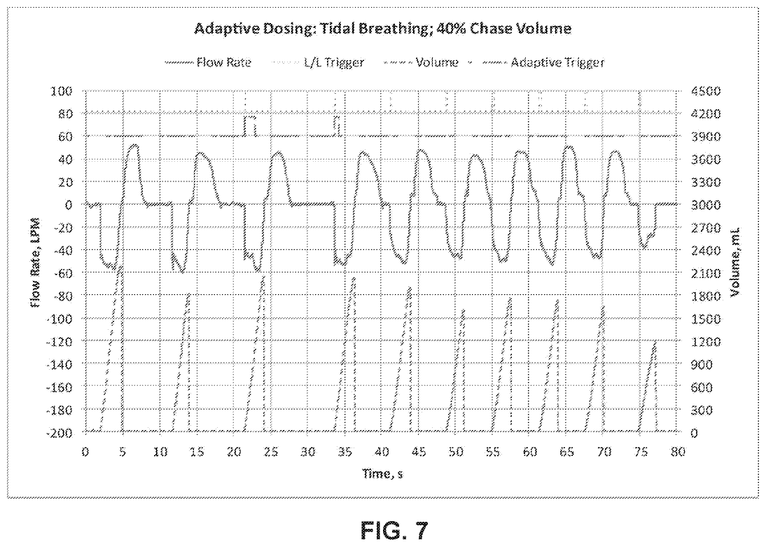

According to another exemplary embodiment, FIG. 7 illustrates a breathing pattern collected from another COPD patient utilizing an inhaler that utilizes the adaptive triggering technique described herein. The breathing style of this patient included strong, regular tidal breathing, with 40% chase volume. As shown in FIG. 7, the patient's breathing cycle is a tidal breathing pattern with large, slow breaths as depicted by the bottom two lines in the graph. The adaptive triggering technique reduces the number inhalations required to complete delivery of the dose from eight as is the case when using the non-adaptive (fixed) trigger technique, to two when using the inventive adaptive triggering technique, as depicted in the line second from the top. Due to large volume breathing cycles, very large shot volumes enable the dose to be completed in two inhalations.

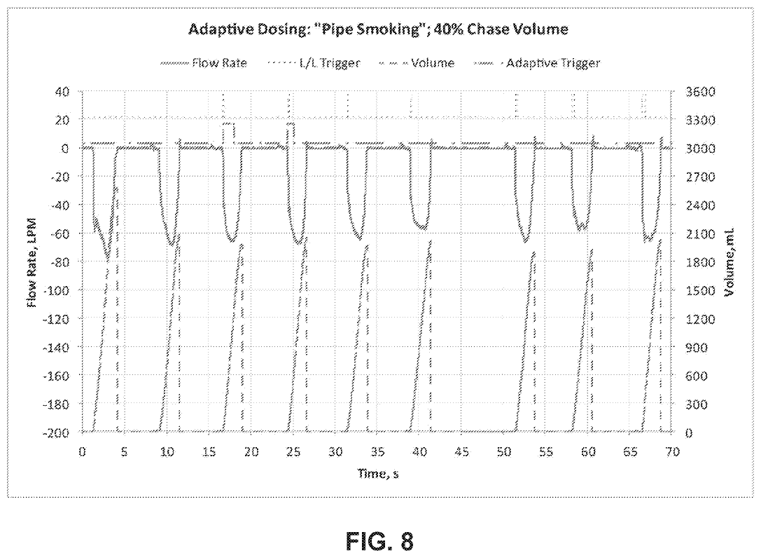

According to another exemplary embodiment, FIG. 8 illustrates a breathing pattern collected from another COPD patient utilizing an inhaler that uses the adaptive triggering technique described herein. The breathing style for the patient involved forced inspiratory maneuvers, indicative of strong, steady pipe smoking. As shown in FIG. 8, the patient's breathing cycles comprise very deep inhalations with strong, steady flow rate as depicted by the bottom two lines in the graph. The adaptive triggering technique reduces the number inhalations required to complete delivery of the dose from eight as is the case when using the non-adaptive (fixed) trigger technique, to two when using the inventive adaptive triggering technique, as depicted in the line second from the top. Due to the characteristics of the patient's breathing cycles, very large shot volumes allow the dose to be completed in two inhalations, thereby decreasing the dose time from about 80 seconds to about 28 seconds.

Although the present embodiments have been described in detail for the purpose of illustration based on what is currently considered to be the most practical and preferred embodiments, it is to be understood that such detail is solely for that purpose and that the embodiments are not limited to the disclosed preferred features, but, on the contrary, is intended to cover modifications and equivalent arrangements that are within the scope of the appended claims. For example, it is to be understood that the features disclosed herein contemplate that, to the extent possible, one or more features of any embodiment can be combined with one or more features of any other embodiment.

What is claimed is:

1. A dry powder inhaler, the dry powder inhaler comprising:
a first chamber configured to hold a dry powder and a gas;
a second chamber directly connected to the first chamber by at least one passageway configured to receive an aerosolized form of the dry powder from the first chamber and delivering the aerosolized dry powder to a user;
a sensor configured to monitor pressure in the second chamber;
a vibrator coupled to the first chamber configured to aerosolize the dry powder and cause the aerosolized powder to move through the passageway whereby to deliver the dry powder from the first chamber to the second chamber as an aerosolized dry powder; and
a vibrator control unit configured to control operation of the vibrator based on the monitored pressure in the second chamber and a predetermined dosing shot volume.

2. The inhaler of claim 1, wherein the vibrator control unit is further configured to:
determine the user's breath cycle and inhalation volume based on the monitored pressure in the second chamber.

3. The inhaler of claim 2, wherein the vibrator control unit is further configured to:
activate the vibrator for a series of delivery shots during inhalation of the user's breath cycle.

4. The inhaler of claim 2, wherein the vibrator control unit is further configured to:
determine whether the inhalation volume of the user's breath cycle is equal to the predetermined dosing shot volume; and
in response to the inhalation volume of the user's breath cycle being equal to the predetermined dosing shot volume, deactivate the vibrator;
in response to the inhalation volume of the user's breath cycle being not equal to the predetermined dosing shot volume, deactivate the vibrator after a predetermined duration of time.

5. The inhaler of claim 4, wherein the vibrator control unit is further configured to:
determine a first inhalation volume for the user's first breath cycle based on the monitored pressure in the second chamber;
determine subsequent inhalation volumes for the user's breath cycles based on the monitored pressure in the second chamber;
calculate the predetermined dosing shot volume based on the first inhalation volume and the subsequent inhalation volumes.

6. The inhaler of claim 5, wherein the predetermined dosing shot is based on a fixed percentage of a total measured volume of the first inhalation volume and adjusted according to subsequent inhalation volumes.

7. The inhaler of claim 8, wherein the fixed percentage is approximately 30-60 percent of a total measured volume.

8. The inhaler of claim 4, wherein the sensor is further configured to monitor flow rate in the second chamber and the vibrator control unit is further configured to:
determine a peak flow rate of the user's breath cycle based on the monitored flow rate in the second chamber.

9. The inhaler of claim 8, wherein the vibrator control unit is further configured to:
determine whether the user's breath cycle has reached the peak flow rate;
in response to the user's breath cycle reaching the peak flow rate, deactivate the vibrator; and
in response to the user's breath cycle not reaching the peak flow rate, continue activation of the vibrator for a predetermined amount of time.

10. The inhaler of claim 8, wherein the determination of the peak flow rate is based on at least one of a rate or magnitude of changes in flow rate and/or volume in the second chamber.

11. The inhaler of claim 3, wherein the vibrator control unit is further configured to:
determine a total shot duration based on a delivery time of each delivery shot of the series of delivery shots; and
terminate the dosing session in response to the total shot duration equaling a predetermined dosing scheme.

12. A method for delivering an adaptive dose of a drug with an inhaler, the method comprising:
holding a dry powder and a gas in a first chamber;
receiving an aerosolized form of the dry powder in a second chamber connected to the first chamber;
delivering the aerosolized dry powder in the second chamber to a user;
monitoring pressure in the second chamber with a sensor;
aerosolizing the dry powder with a vibrator coupled to the first chamber to deliver the dry powder from the first chamber to the second chamber as an aerosolized dry powder; and
controlling operation of the vibrator based on the monitored pressure in the second chamber and a predetermined dosing shot volume.

13. The method of claim 12, wherein the method further includes:
determining the user's breath cycle and inhalation volume based on the monitored pressure in the second chamber.

14. The method of claim 13, wherein the method further includes:
activating the vibrator for a series of delivery shots during inhalation of the user's breath cycle.

15. The method of claim 13, wherein the method further includes:
determining whether the inhalation volume of the user's breath cycle is equal to the predetermined dosing shot volume; and
in response to the inhalation volume of the user's breath cycle being equal to the predetermined dosing shot volume, deactivating the vibrator;
in response to the inhalation volume of the user's breath cycle being not equal to the predetermined dosing shot volume, deactivating the vibrator after a predetermined duration of time.

16. The method of claim 15, wherein the method further includes:
determining a first inhalation volume for the user's first breath cycle based on the monitored pressure in the second chamber;
determining subsequent inhalation volumes for the user's breath cycles based on the monitored pressure in the second chamber;
calculating the predetermined dosing shot volume based on the first inhalation volume and the subsequent inhalation volumes.

17. The method of claim 16, wherein the predetermined dosing shot is based on a fixed percentage of a total measured volume of the first inhalation volume and adjusted according to subsequent inhalation volumes.

18. The method of claim 15, wherein the sensor is further configured to monitor flow rate in the second chamber and the method further includes:
determining a peak flow rate of the user's breath cycle based on the monitored flow rate in the second chamber.

19. The method of claim 18, wherein the method further includes:
determining whether the user's breath cycle has reached the peak flow rate;
in response to the user's breath cycle reaching the peak flow rate, deactivating the vibrator; and
in response to the user's breath cycle not reaching the peak flow rate, continuing activation of the vibrator for a predetermined amount of time.

20. The method of claim 14, wherein the method further includes:
determining a total shot duration based on a delivery time of each delivery shot of the series of delivery shots; and
terminating the dosing session in response to the total shot duration equaling a predetermined dosing scheme.
